(19) United States
(12) Patent Application Publication
(10) Pub. No.: US 2010/0041979 A1
(43) Pub. Date: Feb. 18, 2010

(54) INTERVENTIONAL IMMOBILIZATION DEVICE

(76) Inventor: Raymond D. Harter, Cross Plains, WI (US)

Correspondence Address: FOLEY & LARDNER LLP, 150 EAST GILMAN STREET, P.O. BOX 1497, MADISON, WI, 53701-1497, US

(21) Appl. No.: 12/575,578

(22) Filed: Oct. 8, 2009

Related U.S. Application Data
(63) Continuation of application No. 11/569,631, filed on Nov. 27, 2006.
(60) Provisional application No. 60/575,657, filed on May 28, 2004.

(51) Int. Cl. A61B 5/055

(52) U.S. Cl. 600/411; 600/415

(57) ABSTRACT

Interventional immobilization devices used to immobilize a body part and then, during a medical procedure, orient a medical device to treat located tissue within the body part are provided. The devices are designed to immobilize body tissue while preserving, or substantially preserving, the three-dimensional or volumetric integrity of the immobilized tissue. The device enables real time (RT) imaging-guided interventional (IGI) capabilities when the devices are coupled with medical imaging systems, such as magnetic resonance imaging (MRI) systems.

### CROSS-REFERENCE TO RELATED PATENT APPLICATIONS

This application is a continuation application of U.S. patent application Ser. No. 11/569,631 filed Nov. 27, 2006, which is a National Stage application of PCT/US2005/018695 filed May 26, 2005, which claims priority to U.S. Provisional Patent Application No. 60/575,657, filed May 28, 2004, all of which are incorporated herein by reference in their entirety.

### BACKGROUND

Improved breast cancer patient management is a major societal issue that is receiving growing national attention. Breast cancer patients are requesting efficient diagnosis and care, as well as solutions with better cosmetic and psychological impact. Magnetic resonance imaging (MRI) is an important clinical procedure for the detection and delineation of breast cancers. Although all women can benefit from the increased sensitivity of breast MR imaging, a current candidate for breast NMI is a woman with radio-opaque breasts, for example due to post-operative scarring or augmentation implants. The high sensitivity of MRI allows detection and characterization of breast lesions not seen by other imaging technologies. Once breast lesions are identified, breast MRI can help guide medical procedures such as biopsies.

Unfortunately, current MRI systems are not optimized for breast biopsy. Most current MRI compatible biopsy systems employ plates with a mesh of holes to direct the biopsy needles and, thus, the trajectory is perpendicular to the compression plate with very limited free-hand angulation. Other designs, which use hemispherical guides to position a biopsy gun, require transversing a long path inside the breast to reach a target close to the chest wall, or opposite site to the point of entrance. In many cases, either of these trajectories may not always be optimal.

### SUMMARY

Interventional immobilization devices used to immobilize a body part and then, during a medical procedure, orient a medical device to treat located tissue within the body part are provided. The devices are designed to immobilize body tissue while preserving, or substantially preserving, the three-dimensional or volumetric integrity of the immobilized tissue. The device enables real time (RT) imaging-guided interventional (IGI) capabilities when the devices are coupled with medical imaging systems, such as magnetic resonance imaging (MRI) systems.

Examples of located tissues that may be treated with the present devices include cancerous lesions within a body part, as well as other pathologies. Located tissue may also include any tissue that displays as contrasted tissue during a medical procedure such as MRI. Examples of these tissues include blood vessels, noncancerous lesions, scars, and bone. The interventional immobilization device may be used to immobilize and direct treatment to a variety of body parts, however, some embodiments of the invention make the interventional immobilization device particularly suitable for use in breast MR imaging. For this reason, in the discussion that follows, the device and the methods for its use will be discussed in the context of the immobilization and directed treatment of a breast.

Due to the wide range of breast and chest anatomies (size and shape) and located tissue positions inside the breast, optimal planning of a medical procedure requires both appropriate preparation of the breast, i.e. immobilization, and choice of the trajectory of the intervention of the medical device, i.e. path of insertion. With optimal planning, the proposed device may better facilitate minimally invasive operations, in contrast to fully invasive operations, of the breast. Minimally invasive operations are often associated with minimal scars, faster recovery and better cosmetic effects, all of which are issues of major psychological and societal importance for breast cancer patients, their families and society in general.

In order to facilitate minimally invasive surgery of the breast, the devices provided herein are capable of providing sufficient degrees of freedom to condition the breast and accommodate appropriate trajectories for current and future MR-guided medical procedures in the breast. For example, the present devices allow for the oblique orientation of immobilization and oblique trajectories for medical devices, such as biopsy needles. Oblique orientation of immobilization, as compared to standard medial-lateral or posterior-anterior orientations, and oblique trajectory, as compared to trajectories perpendicular to the compression plane, provide better operation strategies in many cases. Flexibility in accessing the target tissue is pivotal in order to transverse the shortest distance of tissue and reach areas of limited accessibility, like those close to the chest wall, the axilla tail and behind the nipple. Furthermore, appropriate preparation of the breast with oblique immobilization can be useful in relocating augmentation implants in order to obtain the best position for access to a mass.

Medical devices that may be oriented with the interventional immobilization devices include, but are not limited to, tumor ablation devices, such as cryotherapy, photo-laser, direct electrical current, high frequency focused ultrasound and radiofrequency devices; tumor excision devices, such as vacuum assisted biopsy/excision probes; tissue marker placement devices; and drug/chemical delivery devices, including devices used to deliver anesthesia and contrast agents and/or therapeutic agents to a subject.

One embodiment of the present invention provides an interventional immobilization device that comprises a base and at least one curved compression grid plate attached to the base wherein the at least one curved compression grid plate optionally comprises a plurality of apertures. The compression plates are referred to in this embodiment as compression grid plates, because their apertures form a grid, it should be understood that these apertures are not a necessary feature of the plates and that the plates may be more generally referred to as compression plates. The base may be characterized by an upper surface, which may serve as an attachment surface to which the at least one curved compression grid plate is attached and a longitudinal axis extending through the upper surface (e.g., through the center of the base perpendicular to its attachment surface). The curved compression grid plates are generally characterized by an inner surface having a concave cross-section in the plane perpendicular to the longitudinal axis. The inner surface may be concave across its entire cross section or only across a portion of its cross section. In some of the interventional immobilization devices, the at least one curved compression grid plate is capable of a rotational and/or tilting motion with respect to a perpendicular angle with the base. In another embodiment, the at least one curved compression grid plate can be cup shaped.

Some embodiments of the interventional immobilization devices will further comprise probe positioners attached to a rotary track conveyer fit on the base. The rotary track conveyer enables the probe positioner to be rotated on the base in a circular motion around the longitudinal axis of the device. The probe positioner permits positioning of a medical device along the longitudinal axis of the device. The probe positioner includes a probe guide capable of orienting a medical device with respect to a located tissue within a body part. In certain embodiments, the probe positioner moves on the rotary track conveyor in a circular motion on the base in up to a 360-degree angle. In some embodiments, the flexibility in accessing target tissue is achieved, at least in part, by using a design wherein the one or more curved compression plates and the probe positioner are connected to the base in a manner that allows for the independent rotation, about a longitudinal axis running perpendicular to the upper surface of the base, of the one or more compression plates with respect to the probe positioner.

Embodiments may comprise probe positioners with arms adapted to receive the probe guide. The probe guide may comprise a probe pivot, which may be pivotally attached to the arms by a pivotal pin connection. The probe pivot preferentially may move in an angular motion away from, perpendicular to, or toward the base. The probe guide may move along the arms of the probe positioner in a direction perpendicular to the base. In some embodiments of the interventional immobilization device, the probe pivot is adapted to receive a medical device, such as a biopsy needle.

In some embodiments, the interventional immobilization device will be made of a MRI compatible material. Moreover, when an embodiment of the interventional immobilization device is used in a MRI scan, a radiofrequency (RF) coil may be directly attached to the at least one curved compression grid plate. Conversely, in some embodiments, the RF coil may be attached to the base between the at least one curved compression grid plate and the probe positioner. In other embodiments, the RF coils may be integrated into the platform structure that supports the interventional immobilization device and the RF coils.

In yet another embodiment, the interventional immobilization device may comprise a base with a rotary track, a rotary track conveyor fit onto the rotary track, and a probe positioner attached to the rotary track conveyor. In some embodiments, the rotary track conveyor exists within a base that comprises both an inner portion and an outer portion.

Some of the interventional immobilization devices will comprise a base, a rotary track within the base, a rotary track conveyor fit to the rotary track and a probe positioner attached to the rotary track conveyor, wherein the probe positioner comprises a probe guide capable of orienting a medical device based on polar spatial coordinates with respect to located tissue within a body part. The orienting of the medical device may take place through movement of the probe positioner, the probe guide, or both. In some embodiments, one or more motors may control the movement of the probe guide, the probe positioner, and/or the at least one curved compression grid plate. In certain embodiments, the probe positioner may house the motor.

### BRIEF DESCRIPTION OF THE DRAWINGS

FIG. 1 is a perspective view of an embodiment of an interventional immobilization device.

FIG. 2 is a perspective view portraying the attachment of a curved compression grid plate to the platform.

FIG. 3 is a perspective view of the base of the interventional immobilization device.

FIG. 4 is a perspective view of an embodiment of an interventional immobilization device depicting the attachment and structure of a probe positioner.

FIG. 5 is a perspective view of an embodiment of an interventional immobilization device showing the attachment of the probe guide to the probe positioner.

FIG. 6 is a perspective view depicting a motor for controlling the movement of the probe positioner along the arms of a probe guide motor housing.

FIG. 7A is a perspective view illustrating obstruction of the probe guide path by a grid plate member.

FIG. 7B is a perspective view illustrating repositioning of a grid plate member to create an unobstructed probe guide path.

FIG. 8 is a perspective view portraying the positioning of an interventional immobilization device in a breast coil platform configuration.

FIG. 9 is a perspective view depicting a compression grid plate with an integrated RF coil loop.

FIG. 10 is a perspective view depicting a RF coil loop attached to the inner portion of the base of an interventional immobilization device.

FIG. 11 is a schematic diagram depicting a method of using the interventional immobilization device.

FIG. 12 is a perspective view of an alternative embodiment of an interventional immobilization device.

FIG. 13 is a perspective view portraying the attachment of curved compression grid plates to a base ring.

FIG. 14 is a perspective view depicting mating rotary tracks and an attachment mechanism of the base ring and lower coil ring.

FIG. 15 is a cut-away section perspective view depicting the mating rotary tracks and attachment mechanisms of the base ring, rotary track conveyor and lower coil ring.

FIG. 16 is a perspective view showing the attachment of a probe positioner to the rotary track conveyor.

FIG. 17 is a perspective view of the probe guide carriage assembly along with mechanisms for controlling probe guide motion.

FIG. 18 is a perspective view of the probe positioner and a motor for controlling the vertical movement of the probe positioner along probe positioner arms.

FIG. 19 is a perspective view portraying the interventional immobilization device integrated within a customized breast coil and platform structure.

### DETAILED DESCRIPTION

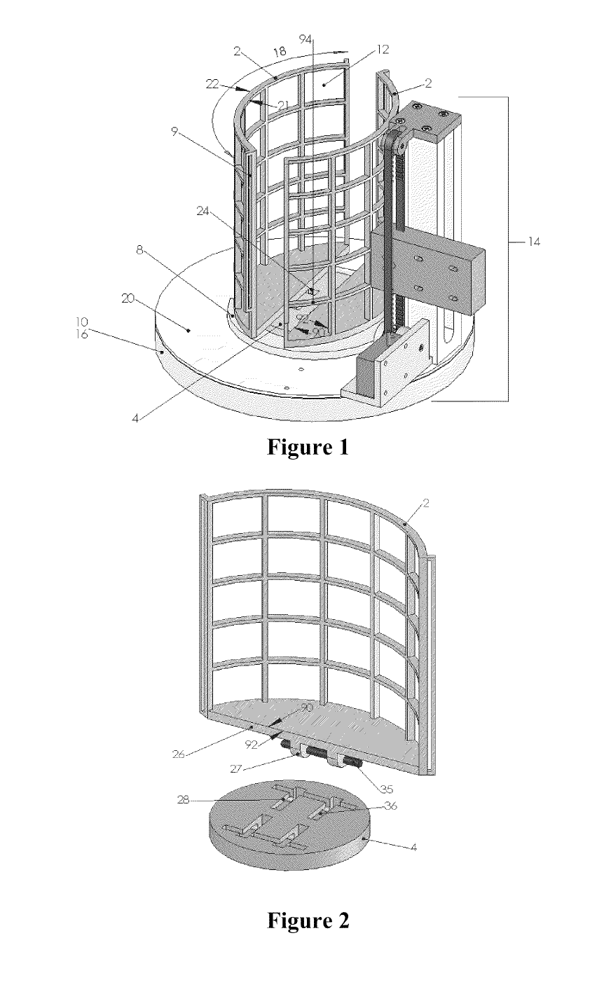

FIG. 1 depicts an embodiment of an interventional immobilization device, which demonstrates two curved compression grid plates (2) and a probe positioner (14) (e.g., a biopsy positioner) attached to a rotary track conveyor (20). The rotary track conveyor (20) fits between an inner portion (8) and outer portion (10) of the base (16). A breast may be received between the curved compression grid plates (2). One of skill in the art will understand that although the embodiment of FIG. 1 shows two curved compression grid plates (2), the number of curved compression grid plates is not so limited. As long as the curved compression grid plates immobilize a breast to a level that satisfies the requirements of the medical procedure, any number of curved compression grid plates, including three, four, or more may be used. In the embodiment of FIG. 1, the curved compression grid plates (2) are connected to a platform (4). Although FIG. 1 demonstrates a removable connection (24) to the platform (4), alternative embodiments may include curved compression grid plates permanently connected to the platform. In the embodiment of FIG. 1, the curved compression grid plates are reversibly attached to the platform. This removable design allows multiple sets of plates with different radii of curvature to be interchanged in order to optimize the plate curvature to the individual patient's anatomical needs.

To satisfactorily position the breast within the interventional immobilization device, the platform (4) may move the curved compression grid plates (2) in the circular motion demonstrated by arrow 18. The entire platform (4) may rotate within the base (16) in up to a 360-degree angle (as denoted by arrow 18). The positioning of the curved compression grid plates (2) allows for accommodation of the different anatomies of the patients encountered, as for example different breast shape and size. As the curved compression grid plates (2) may move in a complete circle, this allows immobilization of the breast in any direction relative to the longitudinal axis of the patient's body. For example, the curved compression grid plates (2) may be positioned so that a path between the curved compression grid plates (2) is not necessarily perpendicular to a line parallel to the spine of the patient. The linear path may form, as a non-limiting example, any angle between 0 and 180 degrees with a line parallel to the spine of a patient.

In certain embodiments, the curved compression grid plates (2) may have slotted edges (9). The slotted edges (9) of the curved compression grid plates (2) allow for fasteners such as Velcro to be looped through the curved compression grid plates (2). The use of a fastener permits increased stability in the positioning of the curved compression grid plates (2). Although the embodiment shown in FIG. 1 demonstrates slotted edges especially adapted for Velcro, in some embodiments, the curved compression grid plates may be additionally stabilized by elastic fasteners or the like. Furthermore, the skilled artisan understands the edges of the curved compression grid plates need not be slotted, but may be of a different configuration which allows stabilization by fasteners such as nuts and bolts and hooks.

The platform (4), through its removable connection (24) to the curved compression grid plates (2) allows the curved compression grid plates (2) to move inward (90) and outward (92) from the center of the platform (94). In this embodiment, the plates may move in the inward and outward motion independently of each other. These movements permit the curved compression grid plates (2) to immobilize many different size breasts. In the embodiment depicted in FIG. 2, the curved compression grid plates (2) are mounted on semi-circular curved compression plate foundations (26), each having a pair of rails (27) extending outwardly from the lower surface of the semi-circular curved compression plate foundation (26). Although the embodiment in FIG. 2 demonstrates two rails, the skilled artisan understands that the number of rails may vary from a single rail to multiple rails. These rails (27) fit into tracks (28) in the platform (4). In this configuration, the rails (27) slide along the tracks (28) to move the curved compression grid plates (2) inward (90) and outward (92).

The curved compression grid plates (2) may also tilt toward (22) or away (21) from each other and the longitudinal axis of the device, made possible by the removable connection (24) of the curved compression grid plates (2) to the platform (4). In certain embodiments, a single or multiple curved compression grid plates may tilt independently of other curved compression grid plates. In the embodiment of FIG. 2, an axle pin (35) is mounted through the rails (27), perpendicular to the sliding direction of motion. This axle pin (35) is captured in a platform slot (36). The platform slot (36) allows the axle pin (35) freedom to slide in and out, and to pivot. The embodiment in FIG. 2 demonstrates the attachment of the semi-circular curved compression plate foundation (26) of the curved compression grid plates (2) to the platform (4). Another embodiment envisions curved compression grid plates attached directly to the platform and without semi-circular curved compression plate foundations.

In some embodiments of the invention, motion of the curved compression grid plates (2) is motor controlled. For example, the platform (4) and/or the semi-circular curved compression grid plate foundations (26) may be mounted to a motor such that the motor controls the rotation and/or translation of the curved compression grid plates (2).

Referring again to FIG. 1, at least one of the curved compression grid plates (2) can contain apertures (12) to provide access for a medical device along the direction defined by the probe positioner (14). The embodiment in FIG. 1 shows apertures (12) in both curved compression grid plates (2); however, not all of the curved compression grid plates (2) need include apertures. Furthermore, although the apertures (12) as shown in FIG. 1 comprise rectangular openings, the shape of the apertures need not be so limited. The apertures in the curved compression grid plates may be any shape that does not affect immobilization and allows access to the breast by the pertinent medical device. These shapes include large squares (like the apertures shown in FIG. 1), as well as finely spaced needle holes such as holes aligned both horizontally or vertically. As yet a further alternative, portions of the curved compression grid plates may be permeable to allow an aperture to be formed as needed. Examples of permeable materials include transparent polymeric sheets. As is known in the art, the proper aperture through which the medical device can be inserted or guided may be discerned by determining which aperture in the curved compression grid plate is closest to the desired entry point. However, surprisingly and unexpectedly, in comparison to the systems known in the art, the combination of certain shaped apertures and curved compression plates provides greater access of a medical device to located tissue.

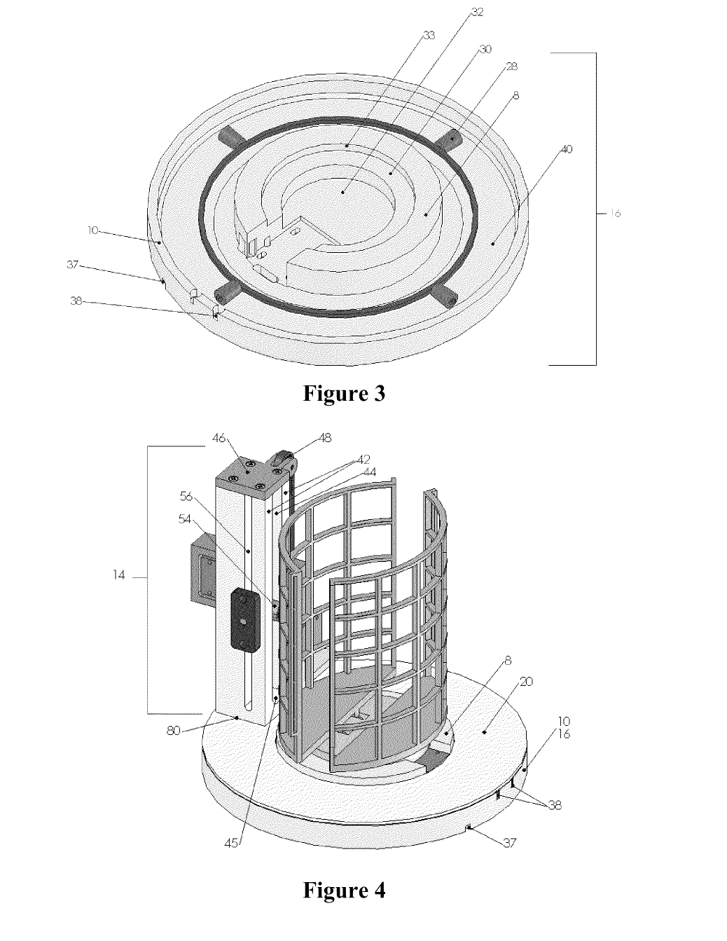

As shown in FIG. 3, the platform rests on the inner base shoulder (30) of the inner portion (8) of the base (16). The inner base shoulder (30) lies above the inner base recess (32). The inner base recess (32) can be used for housing a motor. In the embodiment shown in FIG. 3, the platform (4) described with reference to FIGS. 1 and 2 nests in the inner base recess (32) of the base (16). The platform is held in place by gravity and a close-tolerance mechanical fit between the base counterbore (33) and platform. Other embodiments envision moveable and/or locking retainer tabs, which may hold and/or lock the platform into the base (16). As further shown in FIG. 3, the base (16) comprises a rotary track (40) with roller bearings (28). The rotary track (40) shown in FIG. 3 demonstrates four roller bearings (28); however, any number of roller bearings which allow a rotary track conveyor to move along the rotary track (40) may be used. As shown in FIG. 4, the rotary track conveyor (20) sits on the roller bearings (28) of the rotary track (40) described with reference to FIG. 3. The rotary track conveyor (20) moves in a circular motion along the rotary track between the inner portion (8) and outer portion (10) of the base (16). The embodiment in FIG. 3 also shows a channel (37) in a bottom portion of the base (16). If necessary, this channel (37) may allow for power cord access and proper motor spacing. The skilled artisan understands that the shape and number of channels may be adapted for the specific needs of the particular interventional immobilization device. Also shown in FIG. 3 are two slots (38) in the outer portion (10) of the base. These slots (38) may be used to mount radial load bearings to balance any radial forces that may be exerted on the rotary track conveyor by a motor. The balancing of the radial forces on the rotary track conveyor keeps the rotary track conveyor centered as it rotates. The skilled artisan understands that the number and placement of the slots (38) may be adapted for the specific needs of the particular interventional immobilization device.

FIG. 4 additionally demonstrates a probe positioner (14) permanently attached (80) in a perpendicular orientation relative to the upper surface of the rotary track conveyor (20). The probe positioner (14) demonstrates an appropriate number of degrees of freedom according to the principles of operation of the interventional immobilization device for access to the breast. As understood by one of skill in the art, the permanent attachment (80) of the probe positioner (14) is not meant to be limiting and the present invention may function in the same manner with an attachment made reversible through the use of screws, nuts and bolts, or any other attachment mechanisms known in the art.

In the embodiment of FIG. 4, the probe positioner (14) comprises two rectangular shaped arms (42) divided by a U-shaped slot (44) permanently separating the arms (42). Each of the arms (42) has a longitudinal slot (56) extending most the length of the arm. The longitudinal slots (56) do not extend through the edges of the arm. Although the longitudinal slots (56) shown in FIG. 4 are not horizontally centered within the arms (42), alternative embodiments may encompass horizontally centered slots within the arms. The open-end of the arms (42) not connected by the U-shaped base (45) are connected to each other through a plate (46). The plate (46), which is perpendicular to the arms (42), has a mounted upper pulley (48).

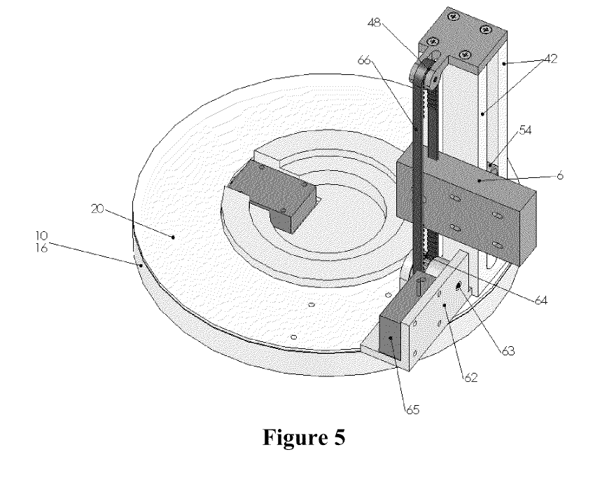

As shown in FIG. 5, a probe motor platform (62) is attached to the probe positioner (14) adjacent to the rotary track conveyor (20). A drive shaft pulley (64) may be mounted on the drive shaft (63) as shown in FIG. 1 or otherwise mechanically coupled to a piezoelectric motor (65) disposed on the probe motor platform (62). A belt (66) can be run between the drive shaft pulley (64) and the mounted upper pulley (48). The belt (66) may be fastened to the probe guide motor housing (6) such that the movement of the belt (66) translates into movement along the arms (42) of the probe guide motor housing (6) and the probe guide (54).

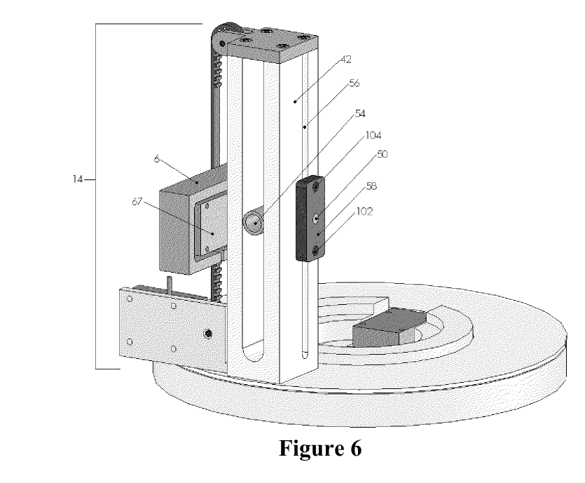

As shown in FIG. 6, a probe guide motor housing (6) is connected to the probe positioner (14) through a pivotal pin connector (50). The pivotal pin connector (50) allows movement of the probe guide motor housing (6) and probe guide (54) along the longitudinal slots (56) in the arms (42) in a direction perpendicular to the plane of the rotary track conveyor. As illustrated in FIG. 6, the pivotal pin connector (50) is composed of three substantially parallel cross beams secured to a beam mount (58) disposed outside one of the arms (42). The upper cross beam (104) and the lower cross beam (102) extend through the longitudinal slots (56) and attach the beam mount (58) to the probe guide motor housing (6). The center cross beam is a pivotal pin connector (50) which passes through the longitudinal slots (56). The pivotal pin connector (50) is secured to the probe guide (54) such that the probe guide (54) rotates with the pivotal pin connector (50). In order to automate the pivoting motion of the probe guide (54), the pivotal pin connector (50) may optionally be mounted to the shaft of a motor, or be otherwise mechanically coupled to a motor held either within the probe guide motor housing (6), held within a different area of the interventional immobilization device, or held outside the parameters of the interventional immobilization device.

The probe guide (54) provides adjustment of the medical device angulation relative to a horizontal plane passing through the longitudinal axis of the interventional immobilization device. When the medical device is a biopsy needle, the probe guide enables a surgeon manually to insert the biopsy needle with an indication of current depth. Advantages to manual insertion include allowing the surgeon to receive perceptible feedback that permits the ascertainment of the density and hardness of the tissue being encountered. Alternatively, a mechanical motor that directs movement to the probe guide may enable a controlled and deliberate insertion of the medical device into the tissue. Certain embodiments of the invention can provide means preventing the probe guide and medical device from moving proximally once inserted into the proper located tissue, thus aiding in maintaining the proper position of the medical device within the located tissue. If the medical device is maintained in the correct location after insertion, the medical device can provide access for other diagnostic and therapeutic tools and treatments.

Although the probe guide (54) shown in FIG. 6 encloses a medical device through friction fitting, the skilled artesian understands that the probe guide may be any apparatus which allows a medical device to be used with the other aspects of the invention. Thus, the probe guide may incorporate a ratcheting or locking feature to prevent inadvertent movement of the medical device. Furthermore, the medical device may be reversibly or rigidly secured to the probe guide. In some embodiments, the probe guide is a universal sleeve that allows for use with an array of medical devices.

Overall movement of the interventional immobilization device allows improvement of the interventional access of the medical device to the tissue of interest. For example, movement of the probe positioner and/or curved compression grid plates may allow precise medical treatment of suspicious tissue hidden behind scar tissue. This movement translates into several advantages associated with the invention, including high flexibility for the definition of the trajectory of insertion of the medical device, flexibility for the definition of the orientation and degree of breast immobilization and an approach to verify the accuracy of positioning by means of MRI visible markers.

Medical devices for use with the interventional immobilization device include any of a number of commercially available biopsy instruments. Alternatively, the medical device could be a therapy probe such as a RF, laser, cryogenic probe or a probe which allows for the localized delivery of drugs. The medical devices may also include catheters, ultrasonic devices, trans-cannular devices, excavating tools, and electrical stimulating devices. Generally, embodiments of the interventional immobilization device are adaptable to accommodate medical devices for performing a variety of trans-cannular or subcutaneous operations.

Once the breast or body part has been received within the curved compression grid plates, the patient may be subjected to a medical procedure such as a MRI scan. NRI permits the identification of suspicious tissue within the breast. If suspicious tissue is detected, the coordinates of any point in the immobilized tissue, including the suspicious tissue may be unambiguously determined relative to a polar coordinate system.

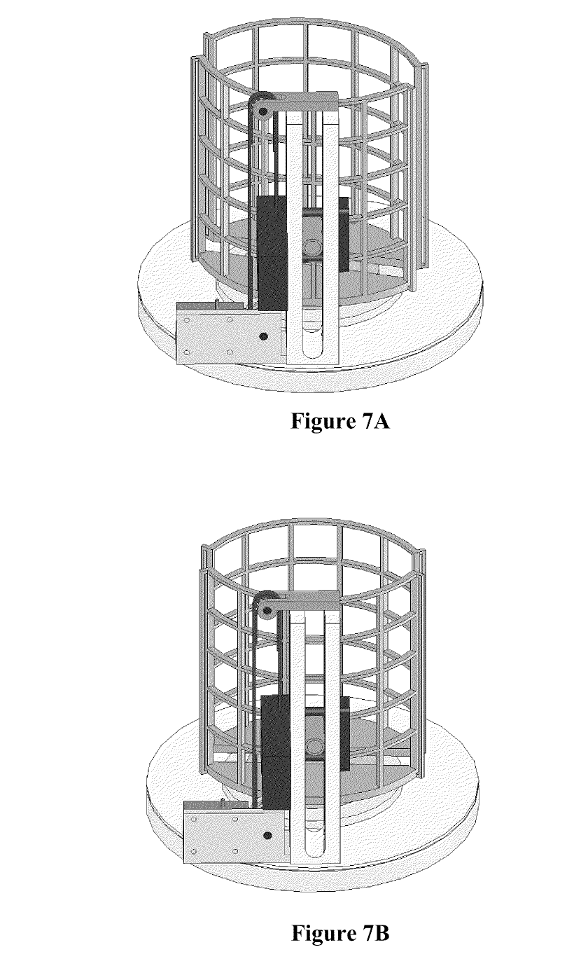

During a breast MRI, at least one of the curved compression grid plates may be repositioned. As shown in FIGS. 7A and 7B, this repositioning may alter the relative position of the tissue within a breast as well as repositioning available entrance routes for invasive medical devices. This advantage is important in breast MR imaging because it allows the breast to be positioned in the best relative position for both medical diagnosis by MR imaging and MR imaging guided medical procedure. Repositioning also allows accurate and rapid medical procedures of breast tissue with a minimum of insertions of a medical device. FIG. 7A illustrates the probe guide path obstructed by one of the bars which form the curved compression grid plates. FIG. 7B illustrates an unobstructed probe guide path following repositioning of the curved compression grid plates shown in FIG. 7A.

During repositioning of the various elements of the embodiment, independent or synchronous controlled motion of the curved compression grid plates and probe positioner is possible. In some embodiments of the invention, the probe positioner and the probe guide will be repositioned during medical procedures such as MRI scans. Certain embodiments allow this movement to be remote controlled. In remotely controlled embodiments, software known in the art may be used to guide the movements. One of skill in the art understands that the type of software used to control movement is not limited and any type of software that works with the device and methods of the present invention may be used. Certain embodiments may employ commercially available software. Other embodiments may employ software custom made for the device and methods of the current invention. In some embodiments, the software may allow movement to be preprogrammed. In alternative embodiments, the software may allow movement to be programmed during the actual MRI procedure. Advantages for movement of the probe positioner, and specifically movement of the probe guide, include allowing minimally invasive changes in medical procedure when the located tissue is in a different position than first believed.

Delivery of both initial and repositioning motion can be accomplished by, but not limited to, the following mechanisms: (a) manual movement, (b) ultrasonic/piezo-electric motors, directly placed on the device; (c) hydraulic actuators, for example pistons or rotary hydraulic motors, directly placed on the device; and (d) a combination of the above depending on the particular motion sought as well as the cost of developing the product. Movement effected by a piezo-electric motor, a motor which converts an electrical field to mechanical strain, is a non-limiting example of how a motor may be used to move the movable parts of the interventional immobilization device. FIG. 5 and FIG. 6 demonstrate the placement of piezo-electric motors (65, 67) on the interventional immobilization device. Because of their small size, piezo-electric motors (65, 67) may fit in areas such as the inner portion of the base, the probe guide motor platform and the probe guide motor housing (6).

Generally, piezo-electric motors have no moving parts other than a finger that protrudes from the end of the motor. This finger vibrates at very high frequency, and the vibration pattern causes the finger tip to move in an elliptical pattern. When this finger is pressed against a ceramic strip that is mounted to a linear motion stage, the finger causes the linear stage to move by nudging the strip along as it makes its elliptical pattern. If the finger is pressed against a ceramic ring or disk that is mounted on an axle or shaft, then rotary motion can be produced by the device following the same principal. When the interventional immobilization device is used with MR scanning, additional methods of motion delivery include non-iron motors, directly placed on the interventional immobilization device or in short distance with flexible drive shafts and electromagnetic motors remotely placed with flexible drive shafts.

Actuator mechanisms of the movable parts can be, but are not limited to: (a) directly, through mechanical coupling of the force/motion transducer to the movable parts; (b) directly, through gearboxes and screw shafts; (c) directly, through gearboxes and timing belts; (d) gearboxes and flexible driving shafts; (e) gearboxes and timing belts; (f) hydraulic pistons; (g) rotary hydraulic motors; and (h) any dictated by the particular design combination of the above.

In some embodiments, when using the interventional immobilization device in MR imaging, the base may provide means for anchoring the interventional immobilization device to a MRI coil. The base of the interventional immobilization device may be attached to an existing breast coil configuration by any of a number of simple, appropriate methods, including, but not limited to, screws, nuts and bolts, cam-type locking clamps, hook-and-latch (Velcro-type) fastening systems, and the like. Adoption of one or more of these fastening alternatives may require minor modification of the existing RF coil platform such as bonding a Velcro strip into place, drilling bolt holes, or the like. Other embodiments of the design may entail providing a custom-fit/integrated breast RF coil “platform.”

It is understood that when the interventional immobilization device is being used for magnetic resonance imaging, the interventional immobilization device will be constructed of a non-magnetic material. Materials of construction of the interventional immobilization device should be non-magnetic to avoid artifacts in the images, such as susceptibility (signal void), distortion of the magnetic field gradients used for localization, and thus inaccuracy in spatial localization. Furthermore, materials of construction should be easily machined to give a particular shape according to the needs of the interventional immobilization device to perform the task described herein, and not easily worn-out. Such materials may include any of a wide variety of MR-compatible engineering plastics, such as polyethylene terephthalate (PET), acrylonitrile butadiene styrene (ABS), polytetrafluoroethylene (PTFE), polyvinyl chloride (PVC), and the like. If necessary, other non-ferrous materials such as aluminum or titanium may also be used for moving parts. Brass can be used for bearings. Preferably, in these embodiments, the interventional immobilization device gives no signal detectable by a MR scanner and minimally affects the homogeneity of the main magnetic field.

In order to view the interventional immobilization device and use the polar coordinates to determine the position of the located tissue, the interventional immobilization device may be made magnetic resonance (MR) visible by embedding or attaching MR visible material. The use of MR visible material allows the desired medical procedure site location to be determined with reference to the MR visible material. The medical device used with the interventional immobilization device preferably also has MR visibility. The MR visible material may encompass any shape, dimension and position as determined by the need to monitor the described MR-guided procedures. The MR visible material may include, but is not limited to, tubes filled with water, gd-DPTA, metal markers or vegetable oil.

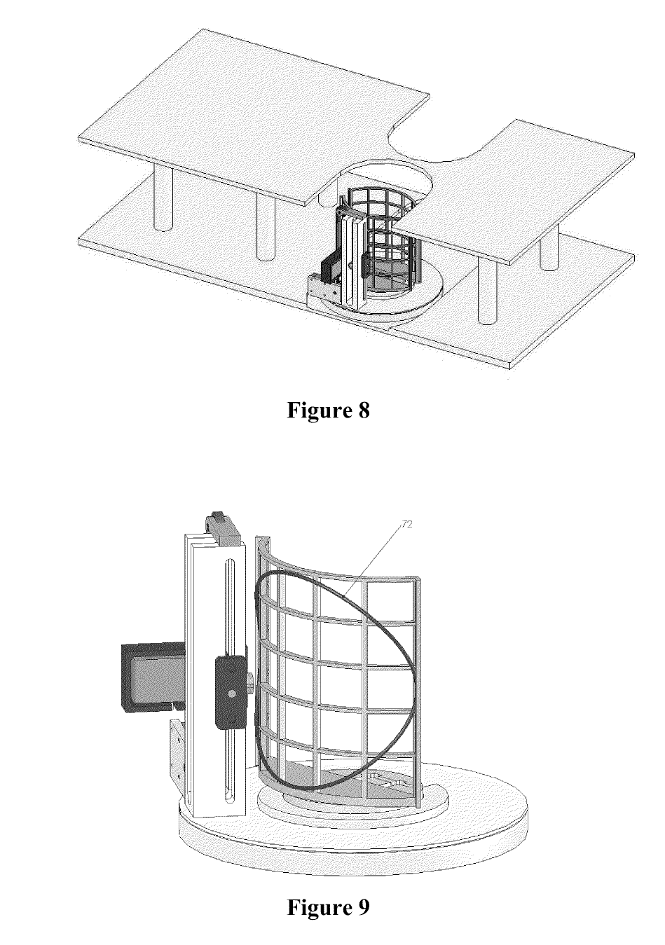

When used in a MRI scan, the interventional immobilization device can be used with commercially available breast imaging RF coils. The breast imaging RF coils are not limiting and any known breast imaging coil where the interventional immobilization device can be modified to fit may be used. In many embodiments, the RF coils will be embedded in breastplates. Some non-limiting examples of breast imaging coils, some of which include RF coils embedded in breastplates, involve those disclosed in Konyer et al., Comparison of MR Imaging Breast Coils, Radiology, Vol. 222 (3): 830-834 (2002), hereby incorporated by reference. FIG. 8 demonstrates an embodiment of the interventional immobilization device inside a typical breast coil platform configuration. As another example, a quadrature RF coil is a relatively conventional coil that may be used with the present invention. Nevertheless, specialized coils that better fit a particular anatomy or task may be used, as they are often beneficial for improved sensitivity of signal detection. In general, fulfillment of the aim of achieving optimal access to the breast dictates the design of the RF coil. As an example of aiming to achieve optimal access, the breast plate may have open sides to allow easier access.

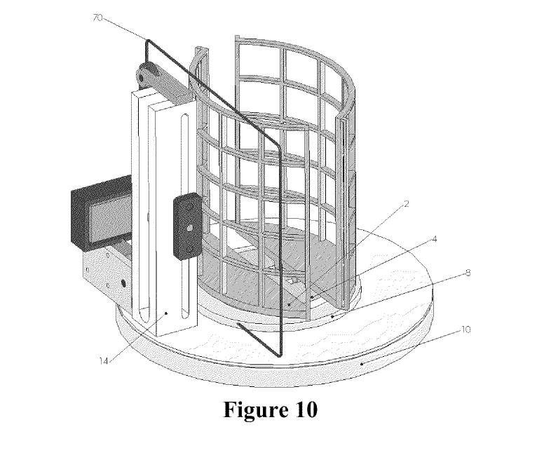

In some embodiments of the present invention, as in the embodiments shown in FIG. 9 and FIG. 10, the RF coil may be attached directly to the interventional immobilization device. Attaching the RF coil directly to the interventional immobilization device may result in increased sensitivity of the MR image. In some embodiments, such as shown in FIG. 9, the RF coil may be attached directly to the curved compression grid plates. In the embodiment of FIG. 9, the shape of the RF coil may vary. Applicable shapes include RF loops (72), RF circles, RF squares and RF rectangles. As shown in FIG. 10, RF coils (70) may alternatively be attached to the interventional immobilization device inner portion (8) of the base between the curved compression grid plates (2) and the probe positioner (14). When attached to the base, the shapes of the RF coils need not be square like that shown in FIG. 10 but instead may be other shapes, including circular and rectangular. Although FIG. 10 shows the RF coil (70) attached to the inner portion (8) of the base, the RF coil may also be attached to the outer portion (10) of the base or to the platform (4).

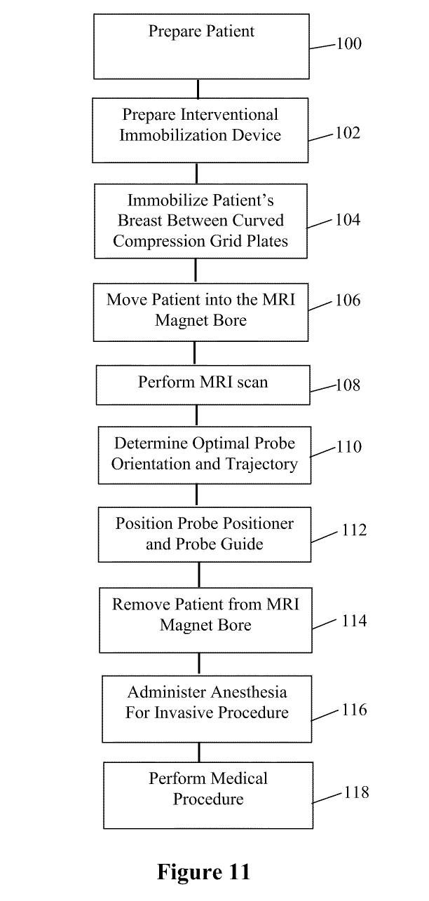

FIG. 11 depicts a sequence of operations, or a method for performing a MRI-guided breast core biopsy in either a closed or open MRI. Following preparation of the patient (100), the interventional immobilization device may be prepared (102). The preparation of the interventional immobilization device (102) may include, but is not limited to, setting-up the interventional immobilization device in or with a RF coil and attaching power and control cables. The patient's breast is immobilized in the interventional immobilization device between the curved compression grid plates (104) and the patient is moved into the MRI magnet bore (106). A MRI scan is performed (108) to stereotopically detect located tissue with reference to a marker that may be on the interventional immobilization device. Using the positional information of the located tissue and other factors that may influence the intervention planning, the physician then determines the optimal orientation and trajectory of the interventional probe (110). Using the positioning capabilities of the interventional immobilization device, the probe positioner and probe guide are positioned (112) at the desired orientation and trajectory for insertion of the medical device into the predetermined treatment site. With real-time imaging capabilities, the physician can observe and verify this positioning movement. Nevertheless, one of skill in the art understands that in some embodiments, the patient may be removed from the MRI before positioning of the interventional immobilization device. For a closed MRI magnet bore, the patient is then removed (114). The removal of the patient is not necessary for an open bore. Anesthesia is administered (116) prior to any invasive medical procedure. In some embodiments, anesthesia may be administered before putting the patient into the MRI magnet bore. A medical procedure can then be performed (118). If a biopsy is performed, after the biopsy, the medical device provides an excellent opportunity to allow other minimally invasive procedures. For example, following a biopsy, a tissue marker may be inserted through the medical device so that subsequent ultrasonic, X-ray, or MRI scans will be able to identify the location of the biopsy.

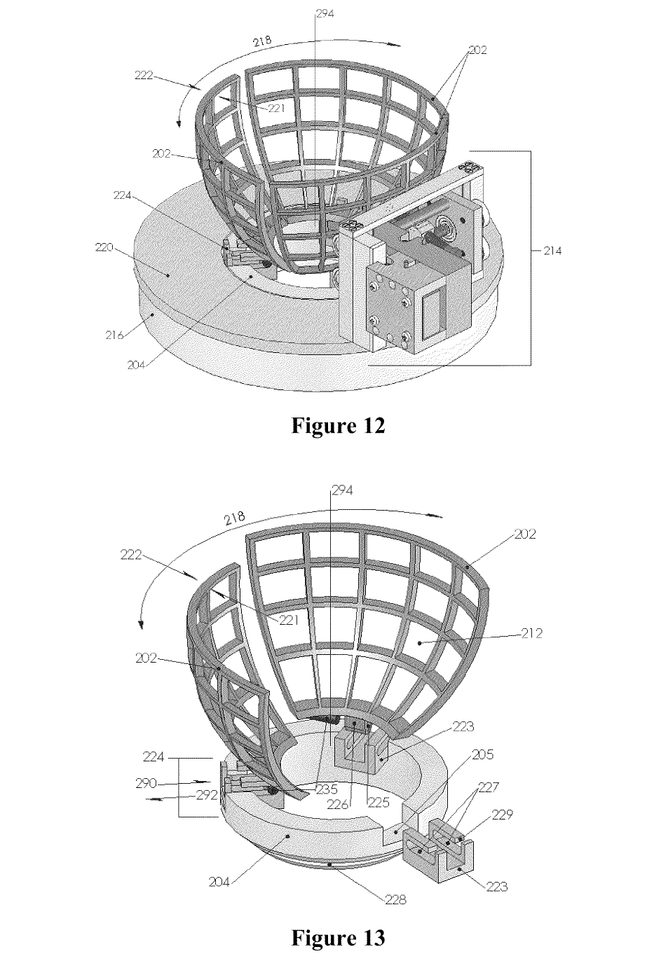

FIG. 12 depicts an alternate embodiment of an interventional immobilization device, which demonstrates three curved compression grid plates (202) and a probe positioner (214) (e.g., a biopsy probe or biopsy needle positioner) attached to a rotary track conveyor (220). In the embodiment shown in FIG. 12, the curved compression grid plates (202) comprise sections of a compound curved, or cup-shaped body, having radii of curvature in two orthogonal axes. A breast may be received between the curved compression grid plates (202). One of skill in the art will understand that although the embodiment of FIG. 12 shows three curved compression grid plates (202), the number of curved compression grid plates is not so limited. As long as the curved compression grid plates immobilize a breast to a level that satisfies the requirements of the medical procedure, any number of curved compression grid plates, including two, four, or more may be used. In the embodiment of FIG. 12, the curved compression grid plates (202) are connected to a base ring (204). Although FIG. 12 demonstrates a removable connection (224) to the base ring (204), alternative embodiments may include curved compression grid plates permanently connected to the base ring. In the embodiment of FIG. 12, the curved compression grid plates (202) are reversibly attached to the base ring (204). This removable design allows multiple sets of plates with different radii of curvature to be interchanged in order to optimize the plate curvature to the individual patient's anatomical needs.

In the embodiment of FIG. 13 the base ring (204), through its removable connection (224) to the curved compression grid plates (202) (shown here with apertures (212)) allows the curved compression grid plates (202) to reversibly move inward (290) and outward (292) from the axis of the base ring (204). In this embodiment, the plates may move in the inward (290) and outward (292) direction independently of each other. These movements permit the curved compression grid plates (202) to immobilize many different size breasts. In the embodiment depicted in FIG. 13, the curved compression grid plates (202) are mounted on a mounting boss (226) attached at the base of the compression grid plates (202). The mounting boss (226) contains a through-hole (225) that accommodates an axle pin (235). The axle pin (235) mounts into parallel slots (227) of a mount block (223), which is attached into a channel (205) of the base ring (204). In this configuration, the axle pins (235) slide along the parallel slots (227) to move the curved compression grid plates (202) inward (290) and outward (292).

The curved compression grid plates (202) may also reversibly tilt toward (222) or away (221) from each other and the longitudinal axis of the device (294). This is made possible by the removable connection (224) of the curved compression grid plates (202) to the base ring (204). In certain embodiments, a single or multiple curved compression grid plates may tilt independently of other curved compression grid plates. In the embodiment of FIG. 13, an axle pin (235) is mounted in the parallel slots (227) of each mount block (223), perpendicular to the sliding direction of motion. The parallel slots (227) allow the axle pin (235) freedom to slide in and out, and to pivot. The embodiment in FIG. 13 depicts a notch (229) in each parallel slot (227), through which the axle pins (235) may be slid to remove and interchange different sets of curved compression grid plates (202) with different radii of curvature in order to optimize the plate curvature to the individual patient's anatomical needs.

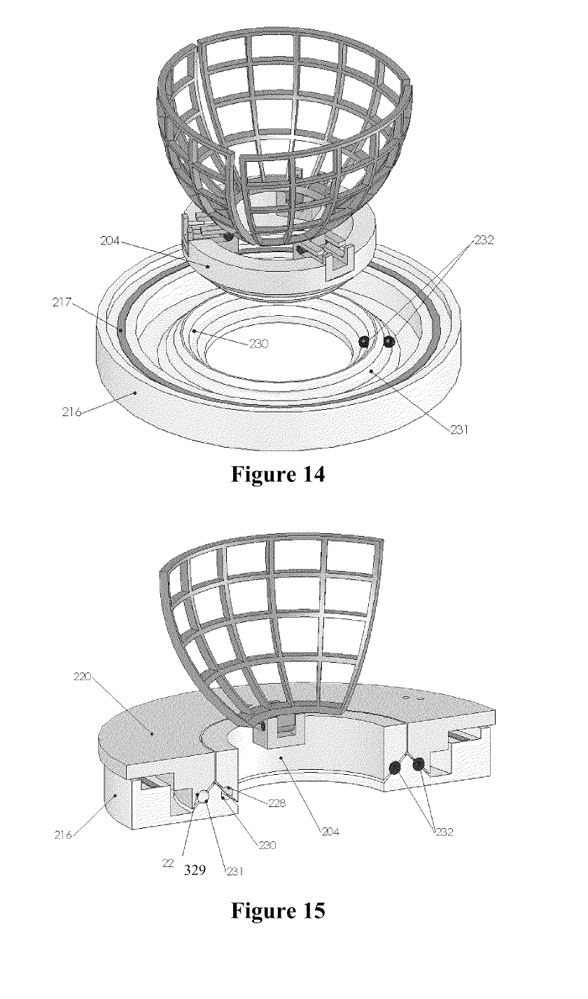

As shown in FIG. 13, the center of the base ring (204) is open to allow the pendant breast to protrude through the base ring (204) if necessary, thereby allowing more flexibility to accommodate the individual patient's anatomical needs. The base ring (204) is also configured with a mating bearing race (228) comprising a rotary track to accommodate free ball bearings for low friction rotary motion and retention of the base ring (204) when assembled with the lower coil ring as shown in FIG. 14 and FIG. 15.

As shown in FIG. 14, the base of the interventional immobilization device may comprise a lower coil ring (216), which serves as an integral part of the RF coil platform structure and houses a coil loop (217) of the RF circuitry, as well as serving as the rotary track and mounting structure for the nested rotary motion stages consisting of the base ring (204) and the rotary track conveyor (220 of FIG. 15). In the embodiment shown in FIG. 14, the rotary track of the lower coil ring (216) comprises dual bearing races, an inner bearing race (230) for mounting the base ring (204) and an outer bearing race (231) for mounting the rotary track conveyor (220). Free ball bearings (232) are placed in the inner bearing race (230) and outer bearing race (231) to enable low friction rotary motion of the base ring (204) and the rotary track conveyor. The free ball bearings (232) also serve as a retaining mechanism to hold the base ring (204) and rotary track conveyor (220) securely once they are pressed into place as shown in the cut away view of FIG. 15. One of skill in the art will recognize that a minimum of three free ball bearings (232) located in each the inner bearing race (230) and the outer bearing race (231) and spaced approximately equally around the circumference of the race will be adequate to serve as a retaining mechanism for the mating part and provide for low-friction rotary motion of the mating part. However, the skilled artisan will also recognize that the maximum number of ball bearings that can be equally spaced around the circumference of the race, without contacting other ball bearings, provides the most secure retention mechanism and the most evenly distributed force load between the mating parts while still enabling low-friction rotary motion between the mating parts. The skilled artisan also recognizes that the proper interference fit between the free ball bearings (232), the inner bearing race (230) and outer bearing race (231) of the lower coil ring (216) and the mating bearing race (228) of the base ring (204) and the mating bearing race (329) of the rotary track conveyor (220) will provide a secure retention mechanism for the mating parts. Proper interference fit will also provide a precise rotary motion of the mating parts with minimal axial motion between the mating parts, minimal tilting and minimal rotational run-out or wobble of the rotating parts.

As shown in the embodiment of FIG. 15, the connection of the base ring (204) to the lower coil ring (216) by means of the inner bearing race (230), mating bearing race (228) and free ball bearings (232) enables the base ring (204), and therefore the curved compression grid plates (202), to reversibly rotate in up to a 360-degree angle with respect to the lower coil ring (216) (as shown by arrow 218 in FIG. 12 and FIG. 13). In some embodiments of the invention, the rotation of the base ring and curved compression grid plates, as well as the sliding in or out, or tilting in or out of the curved compression grid plates may be motor-controlled. For example, the base ring and/or curved compression grid plates may be directly or indirectly coupled to a motor such that the motor controls the rotation and/or translation and/or tilting of the curved compression grid plates. Likewise, the connection of the rotary track conveyor to the lower coil ring by means of the bearing races and free ball bearings enables the rotary track conveyor, and therefore the probe positioner, to reversibly rotate in up to a 360-degree angle with respect to the lower coil ring. In some embodiments of the invention, the rotation of the rotary track conveyor and probe positioner may be motor-controlled. For example, the rotary track conveyor may be directly or indirectly coupled to a motor such that the motor controls the rotation of the rotary track conveyor and probe positioner.

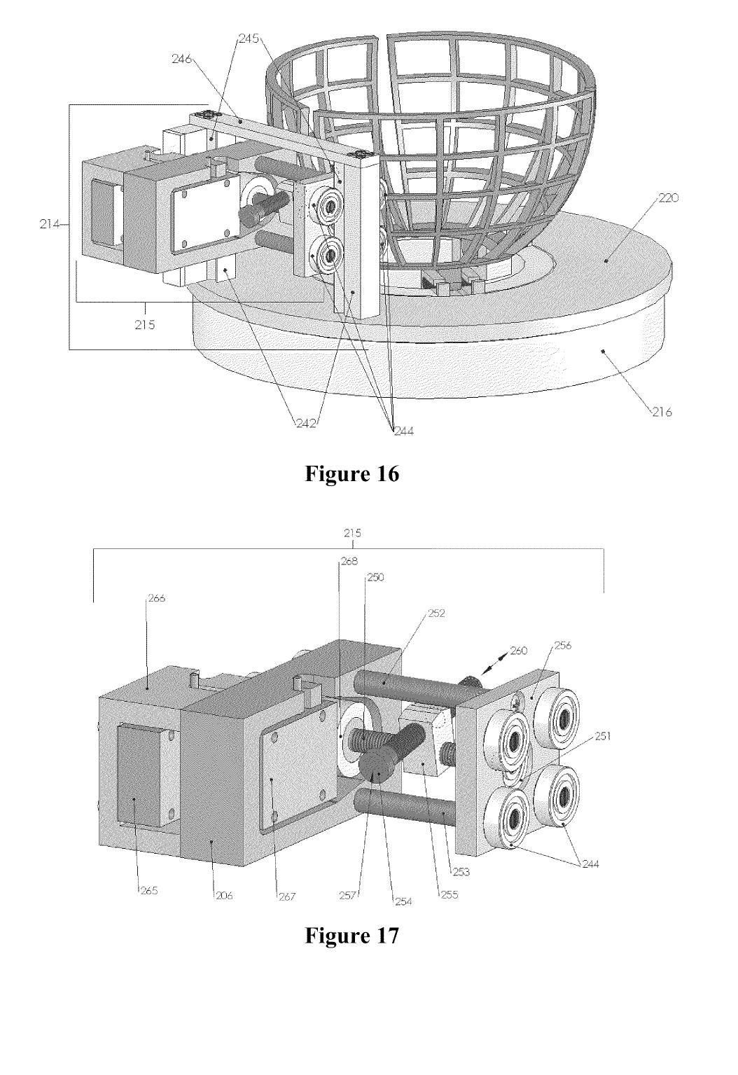

In the embodiment of FIG. 16, a probe positioner (214) is shown attached to the rotary track conveyor (220). The probe positioner (214) comprises two arms (242) attached to the rotary track conveyor (220) and stabilized with a top crossbar (246). The probe positioner (214) also comprises a probe guide carriage assembly (215) that is mounted to the arms (242) with guide roller bearings (244). The guide roller bearings (244) are constrained to roll in guide tracks (245) of the arms (242), thereby enabling precise, reversible vertical motion of the probe guide carriage assembly (215).

As shown in FIG. 17, a pivotal pin connector (250) is also mounted on the probe guide carriage assembly (215) with radial ball bearing cartridges (251) that are mounted at both ends of the pivotal pin connector (250) and fixed into the probe guide motor housing (206) and carriage end plate (256) to enable low friction, precise rotary motion of the pivotal pin connector (250). The pivotal pin connector (250) is designed to accommodate a probe guide (254), wherein the probe guide (254) can be interchangeable. One of skill in the art will understand that although the embodiment of FIG. 17 shows a probe guide (254) with a small center hole (257) designed for receiving and guiding small diameter biopsy or other medical interventional probes, the diameter of the center hole (257) is not so limited. Probe guides (254) with center holes (257) properly sized to receive and guide a wide variety of medical interventional probes may be reversibly interchanged in the probe guide carriage assembly (215). When the interventional immobilization device is used for MR imaging, one of skill in the art will also recognize that the probe guide (254) may also be used as a MR-visible fiducial marker by placing a capillary tube containing MR-visible material (such as gadolinium cheleates, or other MR-visible materials) into the center hole (257). The cylindrical surface of the probe guide (254) contains external screw threads to enable reversible attachment to the probe guide connector block (255), which contains a through hole with mating internal screw threads. The mating screw threads of the probe guide (254) and probe guide connector block (255) also enable secure fixing of the probe guide (254) to the probe guide connector block (255) to prevent inadvertent motion of the probe guide (254), and also enables a useful capability to adjust the depth location of the probe guide (254) to accommodate individual patient anatomical and breast lesion localization needs. By reversibly rotating the probe guide (254) within the probe guide connector block (255), the mating threads cause reversible axial motion of the probe guide (254) as shown by the arrow (260).

In the embodiment shown in FIG. 17, the pivotal pin connector (250) is directly coupled through a ceramic ring (268) to a piezo-electric motor (267) mounted in a probe guide motor housing (206). The piezo-electric motor (267) reversibly controls the rotation of the pivotal pin connector (250), thereby changing the orientation of the probe guide (254) in the vertical plane, through the connection to the pivotal pin connector (250) via the probe guide connector block (255). One of skill in the art will understand that controlled rotation of the pivotal pin connector (250) is not limited to use of a piezo-electric motor (267). The pivotal pin connector (250) may be directly or indirectly coupled to a variety of motor types and designs, including rotary hydraulic or electric motors, or other force transducers, to provide reversibly controlled rotation of the pivotal pin connector (250). Likewise, the probe guide motor housing (206) may be of any design and/or configuration necessary to accommodate the style, type and size of the motor and/or coupling mechanism selected for the design to provide reversibly controlled rotary motion to the pivotal pin connector (250).

The embodiment of FIG. 17 also shows the pivotal pin connector (250) with external screw threads along its shaft. The probe guide connector block (255) is mounted to the pivotal pin connector (250) via a through hole with matching internal screw threads. The mating screw threads of the pivotal pin connector (250) and probe guide connector block (255) enable secure fixing of the probe guide connector block (255) to the pivotal pin connector (250) to prevent inadvertent motion of the probe guide connector block (255), and also enables a useful capability to reversibly adjust the horizontal location of the probe guide connector block (255) along the shaft of the pivotal pin connector (250) to accommodate individual patient anatomical and tumor localization needs. This horizontal location adjustment capability of the probe guide connector block (255) along the shaft of the pivotal pin connector (250) increases the flexible utility of the interventional immobilization device in localization of target breast lesions by providing an additional degree of freedom. The horizontal positioning adjustment of the probe guide connector block (255), and therefore the probe guide (254), axially along the shaft of the pivotal pin connector (250) is achieved by rotating the pivotal pin connector (250) in one direction until the probe guide (254) impacts either the upper carriage pin (252) or lower carriage pin (253) of the probe guide carriage assembly (215), then continuing to rotate the pivotal pin connector (250), forcing the pivotal pin connector threads to rotate within the probe guide connector block (255), thereby causing the probe guide connector block (255) to move axially along the shaft of the pivotal pin connector (250). Likewise, adjustment of the probe guide (254) position in the opposite axial direction along the shaft of the pivotal pin connector (250) is achieved by rotating the pivotal pin connector (250) in the opposite direction until the probe guide (254) impacts either the lower carriage pin (253) or upper carriage pin (252), then continuing rotation of the pivotal pin connector (250) within the probe guide connector block (255) until the desired axial position of the probe guide (254) is achieved.

The pivotal pin connector may also be made with a small center hole along its axis for the entire length of the pivotal pin connector, for a portion of the pivotal pin connector length, or at either or both ends of the pivotal pin connector. When the interventional immobilization device is used for MR imaging, one of skill in the art will recognize that the pivotal pin connector may also be used as a MR-visible fiducial marker by placing a capillary tube containing MR-visible material (such as gadolinium cheleates, or other MR-visible materials) into the pivotal pin connector center hole.

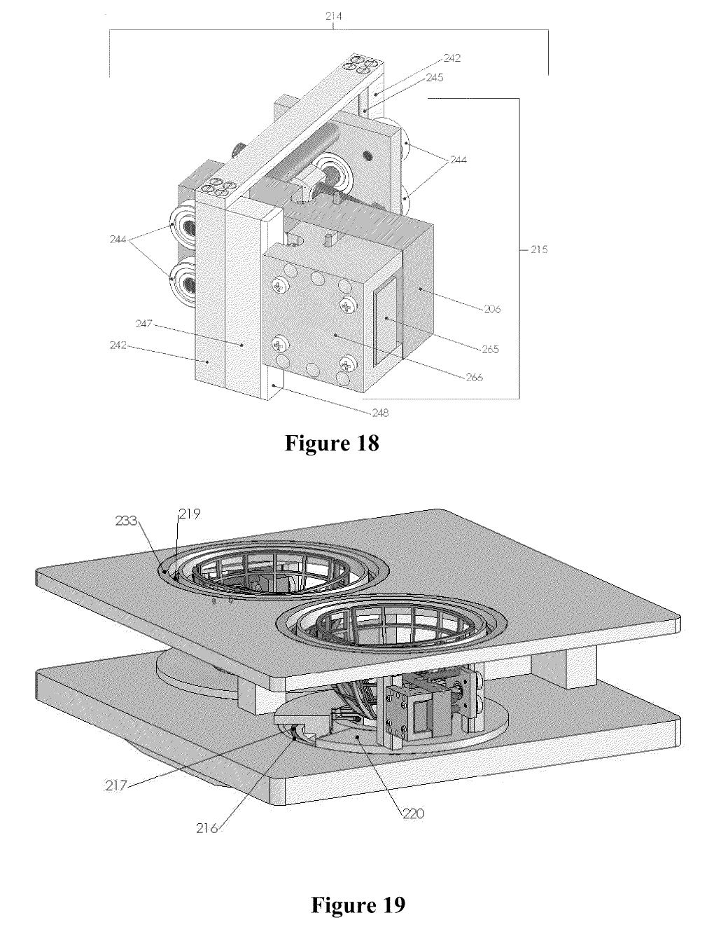

The embodiment of FIG. 18 shows a piezo-electric motor (265) directly coupling the probe guide carriage assembly (215) to an arm (242) of the probe positioner (214) through a spacer block (247) and ceramic strip (248). The piezo-electric motor (265) is attached to the probe guide carriage assembly (215) via a second probe guide motor housing (266), which is attached to the probe guide motor housing (206). In this embodiment of the probe positioner (214), the piezo-electric motor (265) controls the reversible vertical motion of the probe guide carriage assembly (215) along the arms (242). One of skill in the art will understand that controlled vertical motion of the probe guide carriage assembly (215) is not limited to use of a piezo-electric motor (265). The probe positioner carriage assembly may be directly or indirectly coupled to a variety of actuator or motor types and designs, including linear or rotary hydraulic or electric motors, or other force transducers, to provide controlled reversible vertical translation of the probe positioner carriage assembly along the arms. Likewise, the probe guide motor housing may be of any design and/or configuration necessary to accommodate the style, type and size of the motor and/or coupling mechanism selected for the design to provide controlled reversible vertical motion of the probe positioner carriage assembly along the arms.

In some embodiments, when using the interventional immobilization device in MR imaging, the interventional immobilization device may be designed as an integral part of a customized RF receiver coil and platform structure, as shown in FIG. 19. In this embodiment, the lower coil rings (216) house the lower coil loops (217) of the RF circuitry while the upper coil rings (233) house the upper coil loops (219) of the RF circuitry of a Helmholtz pair RF receiver coil for high resolution bilateral breast MR imaging. The lower coil rings (216) and upper coil rings (233) of the interventional immobilization device may be attached to the breast RF coil structure by any of a number of simple, appropriate methods, including, but not limited to, screws, nuts and bolts, cam-type locking clamps, hook-and-latch (Velcro-type) fastening systems, and the like. One of skill in the art will understand that the design of an integrated RF receiver coil and platform structure for use with the intervention immobilization device is not limited to the Helmholtz pair RF receiver coil design, and that the integrated RF receiver coil platform structure may be further customized to accommodate other RF receiver coil designs for use with the interventional immobilization device.

In the embodiments of the interventional immobilization device shown in FIG. 12 through FIG. 19, the nested rotary motion stages (rotary track conveyor and base ring) are attached via their rotary tracks (bearing races) to the lower coil ring via its rotary tracks (bearing races). One of skill in the art will recognize that either one, or both, of these rotary motion stages (rotary track conveyor and base ring) could alternatively be similarly attached to the upper coil ring. For example, the upper coil ring could be designed with a rotary track comprising an internal and/or an external bearing race to accommodate the rotary track(s) of the base ring and/or rotary track conveyor. The rotary track conveyor and/or base ring could then be suspended from the upper coil ring. In the case where the base ring is alternatively suspended from the upper coil ring, the curved compression grid plates could have their mounting boss attached at the top (large radius) edge, rather than at the base (small radius) edge.

In most MRI procedures, a contrast agent is used, which aids in increasing the sensitivity of the scan. An example of how an embodiment of the interventional immobilization device may be used with a contrast agent includes (a) injecting a contrast agent into the breast thereby enabling the contrast agent to spread within tissues of the breast; (b) allowing the contrast agent to reach at least a predetermined level of contrast; and (c) conditioning the breast to restrict the flow of blood into and out of the breast, which increases the persistence of the contrast agent. Following the initial preparation of the area of the breast to be imaged, a MRI technician may diagnose abnormalities using non-invasive procedures. Then, if needed, an interventional procedure may be performed under the observational technique while the contrast agent persists in the area of concern. A contrast persisting technique, made possible by the ability to change the positioning of the curved compression grid plates, can increase the time that the procedure may be performed under adequate observational conditions, while minimizing the amount and number of times that contrast agent needs to be injected.

Movement of the curved compression grid plates may further provide the ability to manipulate the features of the contrast enhancement of the target area in the breast subsequent to infusion of the contrast material. Contrast enhancing features including peak enhancement and duration of the enhancement time window during the “wash out” phase of the contrast agent, may be prolonged if immobilization of the breast by the curved compression grid plates obstructs or limits the clearance rate of the contrast material out of the breast.

In order to facilitate the operation of the present invention, additional degrees of freedom may be added to the interventional immobilization device according to the principles of the invention, i.e. access to a located tissue in a body with a high degree of flexibility in the trajectory of access. Such additional degrees of freedom can be, but are not limited to, adjustment of the height of the at least one curved compression grid plate and angulation of the probe guide in a direction parallel to the direction of the base.

Moreover, the present invention may be further facilitated by designing the base to make it with as low of a height (profile) as possible. This has the major benefit of providing adequate space between the patient surface and the couch, especially when the interventional immobilization device is used with commercially available breast plates. According to certain embodiments, the base can be designed to place all of the motion instrumentation, which can potentially increase the height of the device, outside of the area of operations for the system. Yet, the exact dimensions of the space available will be determined by the design of the system and spatial constraints such as the available space in the MRI scanner.

The interventional immobilization device may be used with real time MRI scanning. In real time MRI, the system displays constantly updated images of the precise location of surgical instruments relative to a located tissue. Because real time MRI may increase the minimalism of the invasiveness of many procedures such as breast biopsies, real time MRI displays a distinct advantage in comparison to conventional MRI techniques. The rapid data acquisition of real time MRI allows for a reduction in scan time, cost, and patient discomfort. For example, in an embodiment that uses the interventional immobilization device with remote controlled movement of the curved compression grid plates and the probe positioner, a patient may need to be put into the MRI scanner only once. If a suspicious located tissue is detected, the interventional immobilization device may be adjusted to perform the medical procedure without pulling the patient out of the MRI scan. Moreover, a second medical procedure in a different location may also be performed without removing the patient from the MRI scanner. Furthermore, because real time MRI allows literal real time viewing of the medical procedure, the interventional immobilization device may be repositioned if the first medical procedure failed to successfully treat all of the suspicious tissue. Once again, real time MRI allows this to be done without removing the patient from the MRI scanner.

Several embodiments of the present invention have potential significant commercial application. In some embodiments, the interventional immobilization device using MRI guidance, both prepares the breast, by setting the degree of compression and orientation, and positions a medical device along a specified trajectory chosen by the MRI technician or physician. If the various movements of the interventional immobilization device are mechanized, the tasks of preparing the breast and positioning the medical device can be performed, without sacrificing high reliability, while the patient remains inside the MRI scanner.

While the present invention has been illustrated by description of several embodiments and while the illustrative embodiments have been described in considerable detail, it is not the intention of the applicant to restrict or in any way limit the scope of the claims to such detail. Additional advantages and modifications may readily appear to those skilled in the art. For example, although MRI is discussed herein as the imaging modality for stereotopically guiding the medical device, embodiments of the present invention may be used with other imaging systems.

Furthermore, although a prone interventional immobilization device is depicted, embodiments of the present invention may include interventional immobilization devices orientated in other manners such as where the patient is treated standing, lying on one side, or supine. In addition, aspects of the present invention have application to diagnostic guided medical procedures on other portions of the body, as well as application in probe positioning utilizing other minimally invasive diagnostic and treatment devices.

While preferred embodiments have been illustrated and described, it should be understood that changes and modifications can be made herein in accordance with ordinary skill in the art without departing from the invention in its broader aspects as defined in the following claims.

What is claimed is:

1. An interventional device comprising:
a platform having an upper surface and configured to rotate about a longitudinal axis running perpendicular to the upper surface of the platform;
a probe positioner mounted to the platform, wherein the probe positioner is configured to rotate about the longitudinal axis independently of a radio frequency coil;
a cup-shaped plate configured to receive a body part, wherein the cup-shaped plate is mounted to the platform; and
the radio frequency coil mounted to the platform, wherein the radio frequency coil is configured to rotate about the longitudinal axis as the platform rotates, and further wherein the radio frequency coil is configured for use with a medical imaging system.

2. The interventional device of claim 1, further comprising a probe guide configured to receive a medical device, wherein the probe guide is connected to the probe positioner in a manner that allows the probe guide to move along the probe positioner in a direction perpendicular to the upper surface of the platform.

3. The interventional device of claim 2, further comprising the medical device mounted in the probe guide.

4. The interventional device of claim 2, wherein the medical device comprises at least one of a biopsy instrument, a therapy probe, a catheter, an ultrasonic device, a trans-cannular device, an excavating tool, an electrical stimulation device, an anesthesia delivery device, a tissue marker placement device, a drug delivery device, a chemical delivery device, and a tumor excision device.

5. The interventional device of claim 4, wherein the therapy probe comprises at least one of a laser ablation probe, a radiofrequency ablation probe, a direct current ablation probe, and a high frequency ultrasound probe.

6. The interventional device of claim 2, wherein the medical device comprises a biopsy needle.

7. The interventional device of claim 2, wherein the medical device comprises a cryo-ablation therapy probe.

8. The interventional device of claim 2, wherein the probe guide is connected to the probe positioner through a pivotal connector, such that the probe guide is configured to pivot relative to a plane which is parallel to the upper surface of the platform.

9. The interventional device of claim 1, wherein the probe positioner moves along a rotary track.

10. The interventional device of claim 1, wherein the interventional device is made of a non-magnetic material.

11. The interventional device of claim 1, wherein the radio frequency coil is mounted to the cup-shaped plate.

12. The interventional device of claim 1, wherein the platform is mounted to a base.

13. The interventional device of claim 1, wherein the cup-shaped plate is configured to rotate independently of the probe positioner.
